(19) United States
(12) Patent Application Publication
(10) Pub. No.: US 2026/0029282 A1
(43) Pub. Date: Jan. 29, 2026

(54) MEASURING DEVICE FOR MEASURING THE TEMPERATURE OF A MEDICAL ELECTRODE

(71) Applicant: Leonhard Lang GmbH, Innsbruck (AT)

(72) Inventor: Daniel FISCHER, Voels (AT)

(21) Appl. No.: 19/347,005

(22) Filed: Oct. 1, 2025

Related U.S. Application Data
(63) Continuation of application No. PCT/AT2024/060029, filed on Feb. 2, 2024.

(30) Foreign Application Priority Data
Apr. 3, 2023 (AT) A 50235/2023

(51) Int. Cl. G01K 13/20; A61B 18/00; A61B 18/16; G01K 1/02; G01K 7/16

(52) U.S. Cl. CPC G01K 13/20; A61B 18/16; G01K 1/026; G01K 7/16; A61B 2018/00791; A61B 2018/167

(57) ABSTRACT

A measuring device for measuring the temperature and/or a change in temperature of a medical electrode is separate from the measuring device, preferably a neutral electrode. The measuring device includes a support and at least two temperature sensors arranged on the support. The at least two temperature sensors are spatially distanced from each other, and an adhesive layer is provided, via which the measuring device can be arranged on a medical electrode, preferably on the upper face of the medical electrode, and the upper face facing away from the skin.

The present application is a continuation of International Application PCT/AT2024/060029 filed on Feb. 2, 2024. Thus, all of the subject matter of International Application PCT/AT2024/060029 is incorporated herein by reference.

### BACKGROUND OF THE INVENTION

The invention relates to a measuring device for measuring a temperature and/or a temperature change of a medical electrode, preferably a neutral electrode, the medical electrode being separate from the measuring device. The measuring device comprises a carrier and at least two temperature sensors arranged on the carrier, an arrangement made up of a medical electrode and such a measuring device, a device for monitoring a temperature of a neutral electrode using such a measuring device, and the use of such a measuring device for measuring a temperature of a medical electrode separate from the measuring device.

Electrosurgery is a widespread method which is used routinely in many applications. In German-speaking countries, electrosurgery is used in about 80% of all surgical procedures. Electrosurgical treatment uses high-frequency electrical currents to cut and coagulate by conducting the currents through human tissue. A neutral electrode is provided to discharge the currents from the body of the patient again.

The neutral electrode cannot heat the skin of the patient by more than 6° C. during tissue ablation or electrosurgical procedures according to the AAMI HF-18 standard. The HF current return is normally concentrated around the edges of the neutral electrode (edge effect), which results in excessive heating at higher current densities. If used improperly or if the neutral electrode becomes detached, a patient may suffer second or third degree burns. It is therefore essential to monitor the neutral electrode to prevent injury to a patient.

In the prior art, so-called split electrodes are often used for this purpose. In these electrodes, a conductive area is divided into at least two sections. If the electrode is not correctly attached or becomes detached in the course of an operation, this can be detected by impedance measurements of the electrode and a corresponding alarm can be triggered. The disadvantage is that the temperature itself cannot be determined.

Solutions to this problem are known from the prior art. For example, US 2013/0158543 discloses a counter electrode having temperature monitoring for electrosurgery. Various exemplary embodiments for implementing temperature monitoring are mentioned here, for example, via a matrix made up of two different conductors using the Seebeck effect or via a layer of ferromagnetic material. These elements are always integrated into the electrode.

However, this makes the production of such an electrode time-consuming and costly, wherein even small differences make a significant difference in the case of mass-produced items such as neutral electrodes.

In addition, such electrodes are not compatible with conventional HF generators for HF surgery. These would therefore have to be replaced.

### SUMMARY OF THE INVENTION

The object of the invention is to at least partially remedy the disadvantages described above and to provide a measuring device which is improved in relation to the prior art, an arrangement made up of a medical electrode and such a measuring device, a device for monitoring a temperature and/or a temperature change of a neutral electrode using such a measuring device, and the use of such a measuring device for measuring a temperature and/or a temperature change of a medical electrode which is separate from the measuring device.

According to the invention, the at least two temperature sensors are locally spaced apart from one another and an adhesive layer is provided, via which the measuring device is arrangeable on a medical electrode, preferably on an upper side of the medical electrode facing away from the skin. This allows a measuring device to be adhesively bonded on a conventional neutral electrode and the temperature of the neutral electrode to be monitored.

Surprisingly, it has been shown that the measurement on the base material of the medical electrode (web, tape, foam) is sufficiently accurate and reliably detects a temperature change between human skin and the gel/conductive layer of the neutral electrode. Any correction factors are set by the temperature sensors themselves and/or by an evaluation unit.

Because the measuring device can be adhesively bonded onto a conventional electrode, cost-effective, conventional neutral electrodes can still be used. If necessary, a measuring device is simply attached to the medical electrode.

Conventional HF generators can also continue to be used; a separate evaluation unit can be provided for the measuring device.

Due to the local spacing apart of the at least two temperature sensors, the temperature of the medical electrode can be measured at two locally spaced-apart points, thus achieving greater reliability.

An arrangement according to the invention comprises a medical electrode, preferably a neutral electrode, and a measuring device, wherein the measuring device is arranged on the medical electrode, preferably on an upper side of the medical electrode facing away from the skin.

A device according to the invention for monitoring a temperature of a neutral electrode comprises a measuring device and an evaluation unit, which is connected or connectable to the measuring device, preferably via a signal line.

In addition, the use of a measuring device for measuring a temperature of a medical electrode separate from the measuring device is also provided. The measuring device is arranged on the medical electrode, preferably on an upper side of the medical electrode facing away from the skin.

The carrier can be configured to be self-adhesive and the adhesive layer is formed by the self-adhesive carrier. This can further simplify the production of a measuring device.

Preferably, the measuring device has 10 to 20, preferably 12 to 16, temperature sensors. Using such a number of temperatures, the temperature of a medical electrode can be measured at essentially all relevant points.

The at least two temperature sensors can be designed as resistance thermosensors, bimetallic sensors, or thermocouples. In principle, however, all suitable types of temperature sensors are conceivable. This also comprises conventional temperature sensors or chips.

Advantageously, the at least two temperature sensors are printed on the carrier. This reduces the space required and also the production effort for a measuring device.

The measuring device can also have at least one conductor for contacting the at least two temperature sensors. Signals from the temperature sensors and/or energy for the temperature sensors can be transported via the at least one conductor. Advantageously, the at least one conductor can also be printed on the carrier.

Preferably, the at least two temperature sensors have an accuracy of less than or equal to 0.5° C., preferably less than or equal to 0.1° C. Particularly in the range from 30 to 45° C., it is advantageous if an accuracy of less than or equal to 0.1° C. is achieved. This ensures, among other things, that a sufficiently accurate temperature measurement can be carried out.

The measuring device can have at least one connection point for connecting a signal line. Signals can be transported from the measuring device to an evaluation unit and vice versa via a signal line.

With regard to an arrangement according to the invention, the measuring device is designed separately from the medical electrode.

Advantageously, the measuring device can be designed as substantially congruent to the medical electrode. This makes it easier to apply a measuring device to a medical electrode. In particular, the correct orientation and arrangement of the measuring device, and therefore of the temperature sensors, on the medical electrode can be ensured.

Particularly preferably, the medical electrode can have at least one contact surface for contacting skin of a patient, and the at least two temperature sensors are arranged in an edge area of the at least one contact surface. Since the HF current return is normally concentrated around the edges (the edge area of the contact surfaces) of the neutral electrode, it is advantageous if the temperature is measured at these points.

With regard to a device, the evaluation unit can be designed to evaluate signals from the measuring device. In the context of the present application, this is to be interpreted in such a way that signals coming from the measuring device (in the case of active temperature sensors) and/or changes occurring due to temperature changes (in the case of passive sensors) are evaluated by the measuring device.

According to one exemplary embodiment, the measuring device can be supplied with energy via the evaluation unit. This makes it easy to ensure the power supply to the measuring device.

Preferably, the evaluation unit has a signaling device, preferably acoustic and/or optical. Such a signaling device can alert a user of the evaluation unit, for example, a surgeon, of an impermissible temperature drop or rise.

For this purpose, advantageously the evaluation unit can be designed to output a signal via the signaling device upon detection of a predetermined temperature change, preferably a temperature change of +/−4° C. This enables early reaction by surgical and medical personnel. If a corresponding connection to an HF generator is provided, it would also be possible to actively intervene in a current flow.

In addition, not only the upward temperature development required by the standard (maximum +6 Kelvin) can be measured, but also a temperature drop. A drop of the temperature can be an indication of an electrode detaching from the human skin, since the ambient temperature in an average operating room (˜20° C.) is significantly lower than the normal skin surface temperature (˜33° C.). A drop in temperature can also be used as an indicator that the electrode is not properly attached.

Preferably, a relative temperature change is measured and an impermissible temperature increase or decrease is detected therefrom. In principle, however, it would also be possible to determine absolute temperatures.

The device can have a measuring cycle of less than or equal to 30 seconds. This ensures that an early warning signal can be given in the event of a warming or a temperature drop.

### BRIEF DESCRIPTION OF THE DRAWINGS

Further details and advantages of the invention will be explained in more detail below with reference to the drawings, in which:

FIG. 1a is a schematic top view of a measuring device,

FIG. 1b is a schematic bottom view of a measuring device,

FIG. 2 is a schematic exploded view of a measuring device,

FIG. 3a is a schematic bottom view of an exemplary embodiment of a measuring device,

FIG. 3b is a schematic bottom view of a further exemplary embodiment of a measuring device,

FIG. 4a is a schematic perspective view of an arrangement from above,

FIG. 4b is a schematic perspective view of an arrangement from below, and

FIG. 5 is a schematic view of a device.

### DETAILED DESCRIPTION OF THE INVENTION

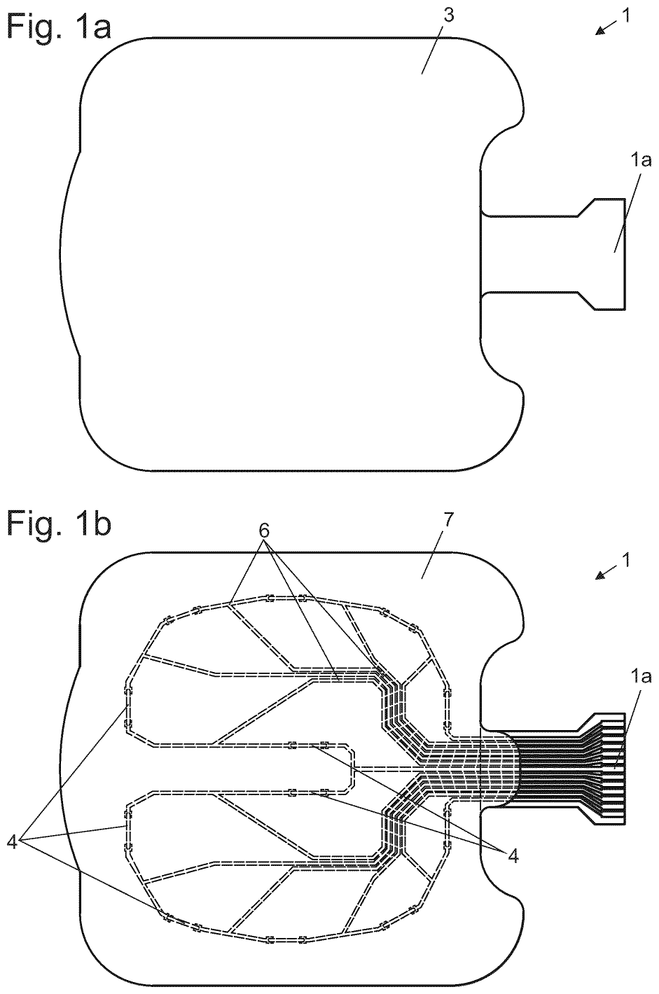

FIG. 1a shows a schematic top view of a measuring device 1 and FIG. 1b shows the corresponding bottom view. In the present exemplary embodiment, this is the application in conjunction with a neutral electrode for HF surgery.

The measuring device 1 has a carrier 3, on which, in the present exemplary embodiment, conductors 6 and temperature sensors 4 are printed. The conductors 6 can be made of silver, for example, and the temperature sensors 4 of carbon. However, other material variants are also conceivable.

The temperature sensors 4 are designed as resistance temperature sensors, i.e. they change their electrical resistance depending on the temperature. In principle, a variety of different sensors are usable in a measuring device 1, for example thermocouples or bimetallic sensors. The temperature sensors 4 and the conductors 6 do not have to be printed on the carrier 3. Conventionally designed conductors 6 and temperature sensors 4 can also be used.

Before and after each temperature sensor 4, the temperature sensors 4 are contacted by a conductor 6, wherein the conductors 6 are led to a connection point 1a. This allows a voltage drop across the temperature sensors 4 to be measured. This can then be used to determine an increase or decrease of the temperature. This is preferably done via a measurement of the relative temperature change. However, it would also be possible to determine absolute temperature values

An adhesive layer 5 is also arranged on the carrier 3. The measuring device 1 can be arranged on a medical electrode 2 via the adhesive layer 5. The carrier 3 can also be designed as self-adhesive. Then the adhesive layer 5 is formed by the self-adhesive carrier 3.

Finally, a protective film 7 is arranged on the adhesive layer 5 to protect the adhesive layer 5 until the measuring device 1 is applied to a medical electrode 2.

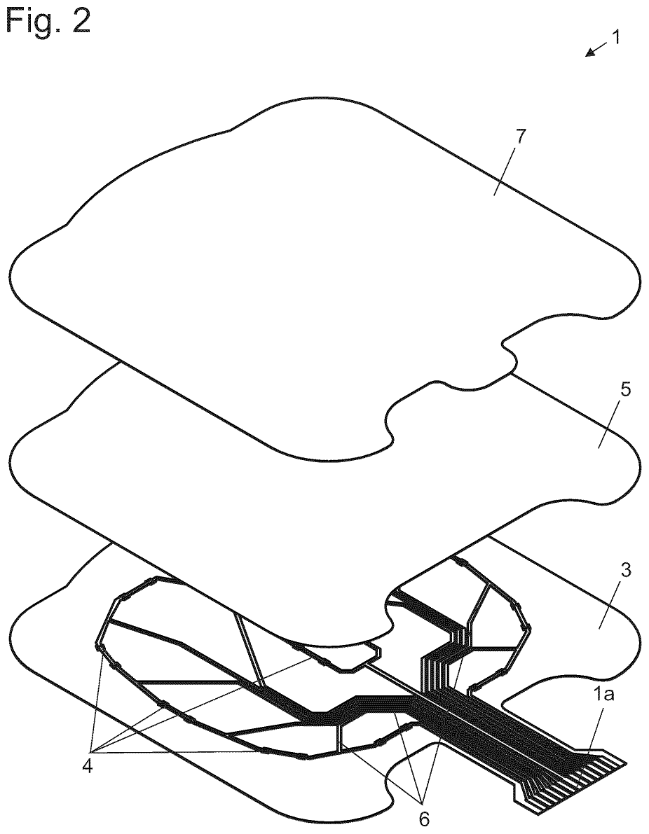

FIG. 2 shows a schematic exploded view of a measuring device 1. The different layers of the measuring device 1 can again be seen.

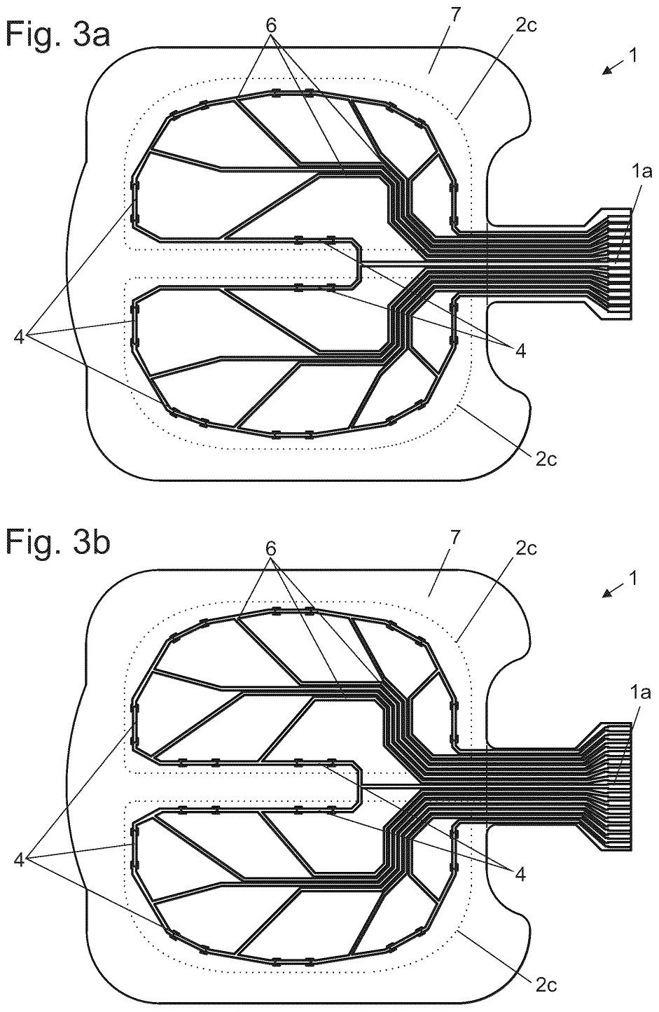

FIG. 3a shows a schematic bottom view of an embodiment of a measuring device 1 and FIG. 3b shows a schematic bottom view of another embodiment of a measuring device 1. The protective film 7 and the adhesive layer 5 are not shown in these figures.

The exemplary embodiments according to FIGS. 3a and 3b differ in the number of temperature sensors 4; in FIG. 3b, two additional temperature sensors 4 are provided.

In addition, it is indicated where contact surfaces 2c of a medical electrode 2 would be located if the measuring device 1 is arranged on a medical electrode 2. It can be seen that the temperature sensors 4 are arranged in an edge area of the contact surfaces 2c. This is because in the edge area, a concentration of the currents returned via the medical electrode 2 occurs, because of which the greatest temperature increase can be expected in this area.

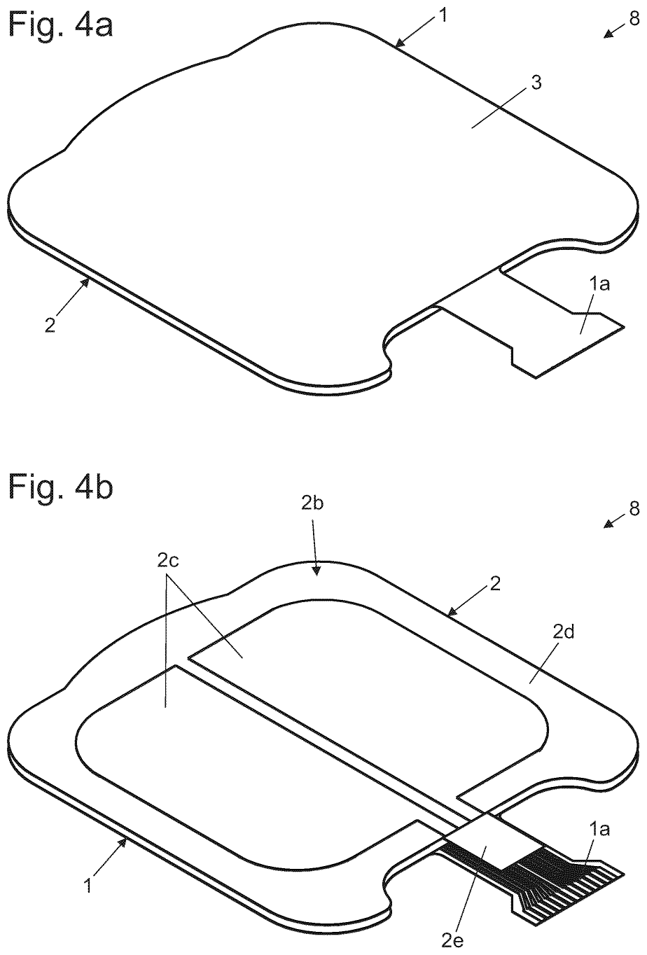

FIG. 4b shows a schematic, perspective view of an arrangement 8 comprising a medical electrode 2 and a measuring device 1 from above, and FIG. 4b shows the same from below.

The measuring device 1 is arranged on an upper side 2a facing away from the skin. The contact surfaces 2c of the medical electrode 2 are arranged on the lower side 2b of the electrode on a carrier 2d.

It can be seen that the measuring device 1 is designed as essentially congruent to the medical electrode 2. This ensures that the measuring device 1 is correctly arranged on a medical electrode 2 and that the temperature sensors 4 are also positioned at the correct points on the medical electrode 2.

The medical electrode 2 also has a connection point 2e for connecting a signal conductor for the medical electrode 2. The medical electrode 2 furthermore comprises an adhesive, using which the medical electrode 2 can be arranged on the skin of a patient. The adhesive can be made electrically conductive in order to establish a conductive connection between the skin of a patient and the contact surfaces 2c of the medical electrode 2. The adhesive is not shown in the present figures. A protective film 7, which can be arranged on the adhesive, is also not shown.

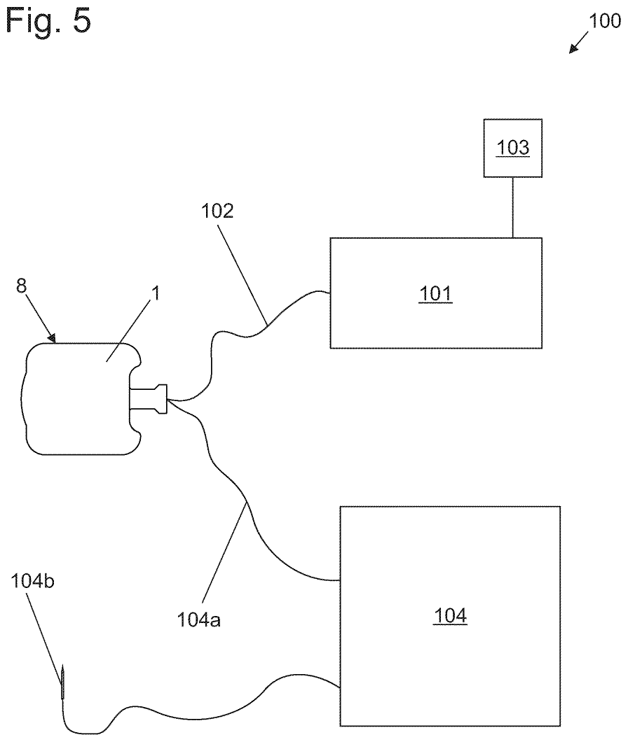

FIG. 5 shows a schematic view of a device 100. An evaluation unit 101 is provided which can evaluate signals from the measuring device 1. The measuring device 1 is connected to the evaluation unit 101 via a signal line 102. The measuring device 1 is furthermore arranged on a medical electrode 2, by which an arrangement 8 is formed.

If the measuring device 1 is designed as shown in FIGS. 1 to 4, the evaluation unit 101 can measure the voltage drop across the individual temperature sensors 4. From this, a relative temperature change and, as a further consequence, a temperature increase or decrease can be detected.

However, it is also conceivable that absolute temperatures are measured and a temperature increase or decrease is detected therefrom.

If the temperature rises or falls by more than a predetermined limiting value, a warning signal can be emitted via a signaling device 103. This can, for example, be of an optical and/or acoustic nature. The limiting value can be set at +/−4° C., for example. This allows early intervention and possible injury to a patient can be prevented.

With appropriate connection to an HF generator 104, it is also conceivable that if the limiting value is exceeded, an active intervention is made in the power supply and, for example, the current flow is reduced.

FIG. 5 shows such an HF generator 104. The HF generator 104 is connected via a cable 104a to a medical electrode 2, via which current can be fed back from a patient to the HF generator 104.

An active electrode 104b is also shown, which is supplied with power by the HF generator 104. Cutting and/or coagulation can be performed via the active electrode 104b by means of electrical current.

### LIST OF REFERENCE SIGNS

1 measuring device
1a connection point
2 medical electrode
2a upper side
2b lower side
2c contact surface
2d carrier
2e connection point
3 carrier
4 temperature sensor
5 adhesive layer
6 conductor
7 protective film
8 arrangement
100 device
101 evaluation unit
102 signal line
103 signaling device
104 HF generator
104a cable
104b active electrode

1. A measuring device for measuring a temperature and/or a temperature change of a medical electrode, preferably a neutral electrode, the medical electrode being separate from the measuring device, wherein the measuring device comprises:
a carrier,
at least two temperature sensors arranged on the carrier, the at least two temperature sensors being locally spaced apart from one another, and
an adhesive layer via which the measuring device is arrangeable on a medical electrode, preferably on an upper side of the medical electrode facing away from the skin.

2. The measuring device according to claim 1, wherein the carrier is configured to be self-adhesive and the adhesive layer is formed by the self-adhesive carrier.

3. The measuring device according to claim 1, wherein the measuring device has 10 to 20, preferably 12 to 16, temperature sensors.

4. The measuring device according to claim 1, wherein the at least two temperature sensors are designed as resistance temperature sensors, bimetallic sensors, and/or thermocouples.

5. The measuring device according to claim 1, wherein the at least two temperature sensors are printed on the carrier.

6. The measuring device according to claim 1, wherein the measuring device has at least one conductor for contacting the at least two temperature sensors.

7. The measuring device according to claim 6, wherein the at least one conductor is printed on the carrier.

8. The measuring device according to claim 1, wherein the at least two temperature sensors have an accuracy of less than or equal to 0.5° C., preferably less than or equal to 0.1° C.

9. The measuring device according to claim 1, wherein the measuring device has at least one connection point for connecting a signal line.

10. An arrangement comprising:
a medical electrode, preferably a neutral electrode, and
the measuring device according to claim 1,
wherein the measuring device is arranged on the medical electrode, preferably on an upper side of the medical electrode facing away from the skin.

11. The arrangement according to claim 10, wherein the measuring device is formed separately from the medical electrode.

12. The arrangement according to claim 10, wherein the measuring device is designed as substantially congruent to the medical electrode.

13. The arrangement according to claim 10, wherein the medical electrode has at least one contact surface for contacting skin of a patient, wherein the at least two temperature sensors are arranged in an edge area of the at least one contact surface.

14. A device for monitoring a temperature and/or a temperature change of a medical electrode, preferably a neutral electrode, comprising:
the measuring device according to claim 1, and
an evaluation unit connected or connectable to the measuring device, preferably via a signal line.

15. The device according to claim 14, wherein the evaluation unit is designed to evaluate signals of the measuring device.

16. The device according to claim 14, wherein the measuring device can be supplied with energy via the evaluation unit.

17. The device according to claim 14, wherein the evaluation unit has a, preferably acoustic and/or optical, signaling device.

18. The device according to claim 14, wherein the evaluation unit is designed to output a signal via the signaling device upon detection of a predetermined temperature change, preferably a temperature change of +/−4° C.

19. The device according to claim 14, wherein the device has a measuring cycle of less than or equal to 30 seconds.

20. A use of the measuring device according to claim 1 for measuring a temperature and/or a temperature change of a medical electrode separate from the measuring device, wherein the measuring device is arranged on the medical electrode, preferably on an upper side of the medical electrode facing away from the skin.
